(19) United States
(12) Patent Application Publication
(10) Pub. No.: US 2006/0206140 A1
(43) Pub. Date: Sep. 14, 2006

(54) Adjustable embolic aneurysm coil

(76) Inventors: Samuel Shaolian, Newport Beach, CA (US); Shawn Moaddeb, Irvine, CA (US); James Hill, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/362,418

(22) Filed: Feb. 23, 2006

Related U.S. Application Data
(60) Provisional application No. 60/656,451, filed on Feb. 24, 2005.

(51) Int. Cl. A61M 29/00

(52) U.S. Cl. 606/200

(57) ABSTRACT

Methods and devices are provided for treatment of an aneurysm within a patient. The devices can be adjusted within the body of a patient in a minimally invasive or non-invasive manner such as by applying energy percutaneously or external to the patient's body. The energy may include, for example, acoustic energy, radio frequency energy, light energy and magnetic energy. Thus, the size and/or shape of the embolic coils can be adjusted to provide optimal filling of the aneurysm. In certain embodiments, the devices include a shape memory material that is responsive to changes in temperature and/or exposure to a magnetic field. A material having enhanced absorption characteristics with regard to a desired heating energy may be used in order to facilitate heating and adjustment of the embolic coil.

### RELATED APPLICATIONS

This application claims the benefit under 35 U.S.C. §119(e) of U.S. Provisional Application No. 60/656,451, filed Feb. 24, 2005, the entirety of which is hereby incorporated by reference herein.

### BACKGROUND OF THE INVENTION

1. Field of the Invention

The present invention relates to methods and devices for treating aneurysms. More specifically, the present invention relates to embolic coils that can be adjusted within the body of a patient.

2. Description of the Related Art

During the last decade or so, endovascular coil embolization has become accepted as an effective method for the treatment of intracranial aneurysms. This technique was initially introduced as a treatment modality for patients at high surgical risk; for example, patients with aneurysms located in the posterior circulation or paraclinoid region, complicating factors such as subarachnoid hemorrhage, comorbid medical conditions, or extreme age. Thus, detachable coil embolization allows for the treatment of aneurysms that were previously considered inoperable. The procedure is less invasive and requires significantly less recovery time than open aneurysmal repair procedures. Blood loss is typically minimal, and local or monitored anesthesia can often be utilized. As the efficacy and safety of this treatment have become more established, indications for aneurysmal coil embolization have increased. This technique is performed as first-line primary therapy in some centers.

One commonly used coiling system for treating aneurysms is the Guglielmi Detachable Coil System (GDC®). In order to treat an aneurysm with GDC® coils, the interventionalist places a microcatheter into the fundus of the aneurysm. Once properly positioned, a coil is inserted through the catheter and into the aneurysm. If the operator finds the coil configuration unsatisfactory, the operator may remove the coil and reposition it within the aneurysm, or, alternatively, choose another size coil. The GDC® system includes a soft platinum coil soldered to a stainless steel delivery wire. When the coil is properly positioned within the findus, a small current, such as 1 mA, is applied to the delivery wire. The current dissolves the stainless steel delivery wire proximal to the platinum coil by means of electrolysis. At the same time, the positively charged platinum theoretically attracts the negatively charged blood elements such as white and red blood cells, platelets, and fibrinogen, and other clotting factors, thus inducing intra-aneurysmal thrombosis. Once electrolysis occurs, the delivery wire can be removed, leaving the coil in place. Additional coils may then, if necessary, be introduced into the aneurysmal sac or fundus. The process is continued until the aneurysm is densely packed with the platinum coils and no longer opacifies during diagnostic contrast injections.

The mechanism by which GDC® coils occlude aneurysms is still being debated. Some empirical observations at surgery on recently coiled aneurysms have lead some to question the theory that the positive charge within the aneurysm during electrolysis induces significant thrombus formation. Coils likely provide immediate protection against rehemorrhage by reducing blood pulsations within the fundus, and sealing the weak portion of the wall or hole. Eventually, organized thrombus does form within the aneurysm and the aneurysm is excluded from the parent vessel by the formation of an endothelialized layer of connective tissue that covers the neck's ostium. This has been demonstrated in experimental dog models and in human autopsy studies.

Long-term success in aneurysmal treatment is dependent on the ability of the coils in controlling the neck of the aneurysm, or fistula. If the coil completely prevents blood flow into the aneurysmal sac, aneurysmal recurrence is unlikely. Coil embolization of small aneurysms with small necks generally has better long-term results than embolization of larger aneurysm with wide necks. Long-term follow-up has shown permanent success in more than 80% of aneurysms treated with coil embolization.

While the indications for GDC® coils are continually expanding as interventionalists become more comfortable and skilled in their placement, coil placement has tended to be most successful in cases of aneurysms with small necks or necks that are smaller in diameter than the maximal aneurysm diameter, as well as aneurysms without significant intrafundal thrombus. Nevertheless, decisions concerning indications for coiling or usually made on a case-by-case basis and few dogmatic rules exist.

However, several concerns remain regarding advances in endovascular treatment using detachable coils. For example, the long-term prognosis for patients status-post aneurysmal coil embolization is not well known. Several articles have reported recanalization of the aneurysm, coil compaction, or subsequent rebleeding during acute short-term follow-up. Also, some detachable platinum coils may not maintain a shape appropriate to effectively occlude the aneurysmal lumen over the long term, which may contribute to aneurysmal recanalization. The percentage of aneurysmal occlusion necessary to prevent recanalization is not well established.

Aneurysms with broad, wide bases, also known as aneurysmal “necks”, are often difficult to coil because if this opening into the aneurysm is too large, the coils have a tendency to slip out of the aneurysm. This “slippage” may cause recanalization as well as potentially dangerous thrombosis of the parent artery or distal embolization.

What is needed is a coil that is more effective in treating larger aneurysms, as well as aneurysms with wide necks. A coil that is adjustable after implantation, that is, able to change shape, if necessary, to better occlude the aneurysmal sac would be extremely useful in this regard. What is also needed is a coil that can be adjusted from outside a patient's body, such as by an external energy source, to obviate the need for the patient to undergo another invasive procedure. Such a coil that is externally adjustable by way of an extrinsic energy source that minimizes heating and potential damage to surrounding neurovascular tissues would also be very advantageous.

### SUMMARY OF THE INVENTION

Thus, it would be advantageous to develop systems and methods for an embolic coil that can be adjusted within the body of a patient in a minimally invasive or non-invasive manner.

In one embodiment, disclosed is a method of treating an aneurysm within a patient, including providing an embolic coil including a shape memory material and having a first size of a dimension of the coil in a first configuration and a second size of the dimension in a second configuration; packing the embolic coil, while the coil is in a first configuration, within an aneurysm; and applying energy from outside the patient's body to the shape memory material of the embolic coil located inside the patient's body, thereby changing the embolic coil from a first configuration to a second configuration.

In another embodiment applying the energy to the embolic coil includes heating the shape memory material of the embolic coil to a predetermined temperature, wherein the shape memory material changes shape in response to being heated to a predetermined temperature.

In another embodiment, heating of the shape memory material includes applying the energy to an energy absorption material in thermal communication with the shape memory material.

In another embodiment, heating of the shape memory material includes applying the energy to an electrically conductive material in thermal communication with the shape memory material, wherein the energy produces a current in the electrically conductive material.

In another embodiment, applying the energy to the embolic coil includes generating a magnetic field outside said patient's body, wherein the magnetic field is configured to change the shape of a shape memory material of the embolic coil.

In another embodiment, the shape memory material includes a ferromagnetic material. In another embodiment, applying the energy includes generating magnetic field energy. In yet another embodiment, applying the energy comprises generating electromagnetic energy.

In another embodiment, applying the energy includes generating mechanical energy. In still another embodiment, applying the energy includes generating acoustic energy. The acoustic energy may be focused ultrasound energy. The acoustic energy may also be high-intensity focused ultrasound energy. In another embodiment, high-intensity focused ultrasound energy is generated with a handheld device. In another embodiment, electromagnetic energy is generated using a handheld device.

In another embodiment, a magnetic field is generated using a magnetic resonance device. Imaging of the embolic coil may also be performed with said magnetic resonance device.

In another embodiment, there is non-invasive monitoring of the sizes of the embolic coil before and after the embolic coil changes from the first configuration to the second configuration. Non-invasively monitoring the sizes of the embolic coil may also include operating a monitoring device comprising at least one of a magnetic resonance imaging device, an ultrasound imaging device, a computed tomography device, and an X-ray device.

In one embodiment, the second size is larger than said first size. In another, the second size is smaller than said first size. In yet another, changing the embolic coil may occur from the second size to a third size of said dimension in a third configuration. The third size is less than said second size in one embodiment. In another, the third size is larger than said second size. In another embodiment, the dimension is a linear dimension.

In another embodiment, the embolic coil further includes an energy-absorbing material over at least a portion of the coil. The coil may also further include a covering extending over at least a portion of said coil.

In another embodiment, disclosed is an adjustable embolic coil, for treating an aneurysm of a patient, including a shape memory material, a first size of a dimension of the coil when said coil is in a first configuration; a second size of said dimension of said coil when said coil is in a second configuration; said coil being changeable from the first configuration to the second configuration in response to an application of energy from outside the patient's body to said shape memory material of said embolic coil, when said coil is located inside said patient's body.

In another embodiment, the coil may further include a first energy-absorbing material extending over at least a portion of said coil. In another embodiment, the embolic coil further includes having a third size of a dimension of said coil in a third configuration, said coil being changeable from the second configuration to a third configuration by applying energy to a shape memory material of said embolic coil.

In yet another embodiment, the coil includes a first energy-absorbing material that absorbs electromagnetic energy. The coil may further include a second energy-absorbing material in another embodiment. The coil may further include a covering that at least partially surrounds the shape memory material. The covering is discontinuous along said embolic coil in some embodiments. In others, the covering has insulative properties. In yet other embodiments, the covering further includes a therapeutic agent. In still other embodiments, the coil includes a thermal conductor coupled to the coil.

For purposes of summarizing the invention, certain aspects, advantages and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

Systems and methods which embody the various features of the invention will now be described with reference to the following drawings:

FIG. 1A is a schematic diagram of an externally-adjustable embolic coil with shape memory portions prior to activation, according to certain embodiments of the invention;

FIG. 1B is a schematic diagram of the externally-adjustable embolic coil of FIG. 1A with shape memory portions shown after activation, according to certain embodiments of the invention;

FIG. 2 is a graphical representation of a length of an embolic coil in relation to the temperature of the coil according to certain embodiments of the invention;

FIG. 3A is a schematic diagram of an adjustable embolic coil with independently-changeable shape memory elements according to certain embodiments of the invention;

FIG. 3B is a schematic diagram of the adjustable embolic coil with independently-changeable shape memory elements of FIG. 3A with one shape memory element activated to its austenitic phase with the other shape memory element remaining in martensitic phase, according to certain embodiments of the invention;

FIG. 3C is a schematic diagram of the adjustable embolic coil of FIG. 3A and FIG. 3B with both shape memory elements shown activated to their austenitic phases, according to certain embodiments of the invention;

FIG. 4A is a schematic diagram of an adjustable embolic coil made of a continuous shape memory member according to certain embodiments of the invention;

FIG. 4B is a schematic diagram of the adjustable embolic coil of FIG. 4A after activation; according to certain embodiments of the invention;

FIG. 5A is a schematic diagram of an adjustable embolic coil covered in part with energy absorption enhancement material, according to certain embodiments of the invention;

FIG. 5B is a cross-sectional view of the adjustable embolic coil of FIG. 5A, according to certain embodiments of the invention;

FIG. 6 is a schematic diagram of an adjustable embolic coil with an insulative covering according to certain embodiments of the invention;

FIG. 7 is a schematic diagram of an adjustable embolic coil comprising one or more thermal conductors according to certain embodiments of the invention.

FIG. 8 is a schematic diagram of an adjustable embolic coil comprising both an insulative covering and thermal conductors according to certain embodiments of the invention.

### DETAILED DESCRIPTION OF THE PREFERRED EMBODIMENT

The present invention involves systems and methods for treating aneurysms with embolic coils. In certain embodiments, an adjustable embolic coil is implanted into the body of a patient such as a human or other animal. The adjustable embolic coil may be implanted percutaneously (e.g., via a femoral artery or vein, or other arteries or veins) as is known to someone skilled in the art. The adjustable embolic coil is packed within an aneurysmal cavity in order to thrombose and occlude the aneurysm, thus preventing rupture of the aneurysmal wall.

The size and shape of the embolic coil can be adjusted postoperatively to compensate for changes in the volume occupied by the coil within the aneurysm. As used herein, “postoperatively” refers to a time after implanting the adjustable embolic coil and closing the body opening through which the adjustable embolic coil was introduced into the patient's body. For example, the adjustable embolic coil may, over time be insufficient to completely occlude the aneurysm, and the aneurysm may recanalize. Recanalization is undesirable in that resumption of blood flow within the aneurysmal sac may cause further weakening and potential rupture of the aneurysmal wall through shear forces. Thus, the size of the adjustable embolic coil may need to be increased to reocclude the entire aneurysm. As another example, the adjustable embolic coil may begin protruding into the lumen of a parent artery (especially in the case of aneurysms with a wide neck), which may promote undesirable thrombosis of the parent artery or embolization of a downstream vessel, potentially causing cerebral ischemia or infarction. Thus, the size of the adjustable embolic coil may need to be decreased postoperatively to ensure confinement of the embolic coil to the aneurysmal cavity.

In certain embodiments, the embolic coil comprises a shape memory material that is responsive to changes in temperature and/or exposure to a magnetic field. Shape memory is the ability of a material to regain its shape after deformation. Shape memory materials include polymers, metals, metal alloys and ferromagnetic alloys. The embolic coil is adjusted by applying an energy source to activate the shape memory material and cause it to change to a memorized shape. The energy source may include, for example, radio frequency (RF) energy, x-ray energy, microwave energy, ultrasonic energy such as focused ultrasound, high intensity focused ultrasound (HIFU) energy, light energy, electric field energy, magnetic field energy, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the embolic coil is selectively heated using short pulses of energy having an on and off period between each cycle. The energy pulses provide segmental heating which allows segmental adjustment of portions of the embolic coil without adjusting the entire coil.

In certain embodiments, the embolic coil includes an energy absorbing material (also referred to herein as energy absorbing enhancement material) to increase heating efficiency and localize heating in the area of the shape memory material. Thus, damage to the surrounding tissue is reduced or minimized. Energy absorbing materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like.

In other embodiments, thin film deposition or other coating techniques such as sputtering, reactive sputtering, metal ion implantation, physical vapor deposition, and chemical deposition can be used to cover portions or all of the embolic coil. Such coatings can be either solid or microporous. When HIFU energy is used, for example, a microporous structure traps and directs the HIFU energy toward the shape memory material. The coating improves thermal conduction and heat removal. In certain embodiments, the coating also enhances radio-opacity of the embolic coil. Coating materials can be selected from various groups of biocompatible organic or non-organic, metallic or non-metallic materials such as Titanium Nitride (TiN), Iridium Oxide (Irox), Carbon, Platinum black, Titanium Carbide (TiC) and other materials used for pacemaker electrodes or implantable pacemaker leads. Other materials discussed herein or known in the art can also be used to absorb energy.

In addition, or in other embodiments, fine conductive wires such as platinum coated copper, titanium, tantalum, stainless steel, gold, or the like, are wrapped around the shape memory material to allow focused and rapid heating of the shape memory material while reducing undesired heating of surrounding tissues.

In certain embodiments, the energy source is applied surgically either during implantation of the coil or at a later time. For example, the shape memory material can be heated during implantation of the embolic coil by touching the embolic coil with a warm object. As another example, the energy source can be surgically applied after the embolic coil has been implanted by percutaneously inserting a catheter into the patient's body and applying the energy through the catheter. For example, RF energy, light energy or thermal energy (e.g., from a heating element using resistance heating) can be transferred to the shape memory material through a catheter positioned on or near the shape memory material. Alternatively, thermal energy can be provided to the shape memory material by injecting a heated fluid through a catheter or circulating the heated fluid in a balloon through the catheter placed in close proximity to the shape memory material. As another example, the shape memory material can be coated with a photodynamic absorbing material which is activated to heat the shape memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the embolic coil. The subcutaneous removable electrode allows more efficient coupling of energy to the implant with minimum or reduced power loss. In certain embodiments, the subcutaneous energy is delivered via inductive coupling.

In other embodiments, the energy source is applied in a non-invasive manner from outside the patient's body. In certain such embodiments, the external energy source is focused to provide directional heating to the shape memory material so as to reduce or minimize damage to the surrounding tissue. For example, in certain embodiments, a handheld or portable device comprising an electrically conductive coil generates an electromagnetic field that non-invasively penetrates the patient's body and induces a current in the embolic coil. The current heats the embolic coil and causes the shape memory material to transform to a memorized shape. In certain such embodiments, the embolic coil also comprises an electrically conductive coil wrapped around or embedded in the memory shape material. The externally generated electromagnetic field induces a current in the embolic coil's coil, causing it to heat and transfer thermal energy to the shape memory material.

The electromagnetic field may utilize direct, rotating, or alternating current. Preferably, the current is alternating current. A time varying magnetic field may be produced by an electromagnetic with a current, preferably alternating current, between 0.0001 Hz to 1000 MHz, preferably 10 Hz to 100 KHz, more preferably 15 KHz to 25 KHz. The alternating current may be modulated, and may also include amplitude, frequency, or phase modulation. In certain embodiments, a time varying magnetic field is produced by one or more electromagnets driven with modulated alternating current sources with controlled phase relationships. In other embodiments, the modulated alternating current sources have controlled phase relationships. In some embodiments, magnets used are permanent magnets that may be mechanically displaced. The mechanical displacement may, for example, an oscillatory or a resonant motion. Furthermore, the time varying magnetic field may be produced by imposing a high frequency magnetic field on one or more low frequency magnetic fields so as to displace the field lines. In other embodiments, the time varying magnetic field is produced by imposing one or more high frequency magnetic fields of a specific phase relationship on one or more low frequency magnetic fields of specific phase relationship so as to displace the field lines. In still other embodiments, a feedback system may provide for regulation and control of the magnetic field intensity, or device temperature. Other embodiments may also include a control system to provide a means of modulating the field such that acquisition of images via ultrasound, fluoroscopy, or other means is enabled. The imaging may be real-time or quasi-real time to allow for viewing of the embolic coil during actuation. The control system may also provide a means of accumulating maximum SAR dosage information and preventing excessive exposure over time.

In certain other embodiments, an external HIFU transducer focuses ultrasound energy onto the implanted embolic coil to heat the shape memory material. In certain such embodiments, the external HIFU transducer is a handheld or portable device. The terms “HIFU,” “high intensity focused ultrasound” or “focused ultrasound” as used herein are broad terms and are used at least in their ordinary sense and include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in certain such embodiments, the focused ultrasound is not destructive to the patient's cardiac tissue. In certain embodiments, HIFU includes acoustic energy within a frequency range of approximately 0.5 MHz and approximately 30 MHz and a power density within a range of approximately 1 W/cm² and approximately 500 W/cm².

In certain embodiments, the embolic coil comprises an ultrasound absorbing material or hydro-gel material that allows focused and rapid heating when exposed to the ultrasound energy and transfers thermal energy to the shape memory material. In certain embodiments, a HIFU probe is used with an adaptive lens to compensate for heart and respiration movement. The adaptive lens has multiple focal point adjustments. In certain embodiments, a HIFU probe with adaptive capabilities comprises a phased array or linear configuration. In certain embodiments, an external HIFU probe comprises a lens configured to be placed on a patient's skull to improve acoustic window penetration and reduce or minimize issues and challenges regarding passing through bones. In certain embodiments, HIFU energy is synchronized with an ultrasound imaging device to allow visualization of the embolic coil implant during HIFU activation. In addition, or in other embodiments, ultrasound imaging is used to non-invasively monitor the temperature of tissue surrounding the embolic coil by using principles of speed of sound shift and changes to tissue thermal expansion.

In certain embodiments, non-invasive energy is applied to the implanted embolic coil using a Magnetic Resonance Imaging (MRI) device. In certain such embodiments, the shape memory material is activated by a constant magnetic field generated by the MRI device. In addition, or in other embodiments, the MRI device generates RF pulses that induce current in the embolic coil and heat the shape memory material. The embolic coil can include an MRI energy absorbing coating to increase the efficiency and directionality of the heating. Suitable energy absorbing materials for magnetic activation energy include particulates of ferromagnetic material. Suitable energy absorbing materials for RF energy include ferrite materials as well as other materials configured to absorb RF energy at resonant frequencies thereof.

In certain embodiments, the MRI device is used to determine the size of the implanted embolic coil before, during and/or after the shape memory material is activated. In certain such embodiments, the MRI device generates RF pulses at a first frequency to heat the shape memory material and at a second frequency to image the implanted embolic coil. Thus, the size of the embolic coil can be measured without heating the coil. In certain such embodiments, an MRI energy absorbing material heats sufficiently to activate the shape memory material when exposed to the first frequency and does not substantially heat when exposed to the second frequency. Other imaging techniques known in the art can also be used to determine the size of the implanted ring including, for example, ultrasound imaging, computed tomography (CT) scanning, X-ray imaging, or the like. In certain embodiments, such imaging techniques also provide sufficient energy to activate the shape memory material.

In certain embodiments, imaging and resizing of the embolic coil is performed as a separate procedure at some point after the embolic coil as been surgically implanted into an aneurysm. However, in certain other embodiments, it is advantageous to perform the imaging after the embolic coil has been placed, but before the introducer catheter apparatus has been removed form the blood vessel. If the amount of filling of the aneurysm is deemed insufficient after implantation of the embolic coil, energy from the imaging device (or from another source as discussed herein) can be applied to the shape memory material so as to better occlude the aneurysm. Additionally, more embolic coils can be inserted as well. Thus, the success of the embolic coil implantation can be checked and corrections can be made, if necessary, before catheter removal.

As discussed above, shape memory materials include, for example, polymers, metals, and metal alloys including ferromagnetic alloys. Exemplary shape memory polymers that are usable for certain embodiments of the present invention are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, each of which are hereby incorporated by reference herein. Shape memory polymers respond to changes in temperature by changing to one or more permanent or memorized shapes. In certain embodiments, the shape memory polymer is heated to a temperature between approximately 38 degrees Celsius and approximately 60 degrees Celsius. In certain other embodiments, the shape memory polymer is heated to a temperature in a range between approximately 40 degrees Celsius and approximately 55 degrees Celsius. In certain embodiments, the shape memory polymer has a two-way shape memory effect wherein the shape memory polymer is heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids such as fluorocarbons and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Exemplary shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other.

In certain embodiments, the shape memory alloy is processed to form a memorized shape in the austenite phase in the form of a coil or coil portion. The shape memory alloy is then cooled below the M_{f} temperature to enter the martensite phase and deformed into a larger or smaller coil. For example, in certain embodiments, the shape memory alloy is formed into a coil or coil portion that is larger than the memorized shape to better improve filling of an aneurysm with coil. In certain such embodiments, the shape memory alloy is sufficiently malleable in the martensite phase to allow a user such as a physician to adjust the length of the ring in the martensite phase by hand to achieve a desired fit for a particular aneurysm. After the embolic coil is packed within the aneurysmal sac, the length of the coil can be adjusted non-invasively by heating the shape memory alloy to an activation temperature (e.g., temperatures ranging from the Aₛ temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the embolic coil to change shape can be selected and built into the embolic coil such that collateral damage is reduced or eliminated in tissue adjacent the embolic coil during the activation process. Exemplary A_{f} temperatures for suitable shape memory alloys range between approximately 45 degrees Celsius and approximately 70 degrees Celsius. Furthermore, exemplary Mₛ temperatures range between approximately 10 degrees Celsius and approximately 20 degrees Celsius, and exemplary M_{f} temperatures range between approximately −1 degrees Celsius and approximately 15 degrees Celsius. The size of the embolic coil can be changed all at once or incrementally in small steps at different times in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Conn.). In certain embodiments, an exemplary Rₛ temperature range is between approximately 30 degrees Celsius and approximately 50 degrees Celsius, and an exemplary R_{f} temperature range is between approximately 20 degrees Celsius and approximately 35 degrees Celsius. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the body. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (Neodynium Iron Boron), SmCo (Samarium Cobalt), ferrite and/or AlNiCo (Aluminum Nickel Cobalt) particles.

Thus, an embolic coil comprising a ferromagnetic shape memory alloy can be implanted in a first configuration having a first shape and later changed to a second configuration having a second (e.g., memorized) shape without heating the shape memory material above the Aₛ temperature. Advantageously, nearby healthy tissue is not exposed to high temperatures that could damage the tissue. Further, since the ferromagnetic shape memory alloy does not need to be heated, the size of the embolic coil can be adjusted more quickly and more uniformly than by heat activation.

Exemplary ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, embolic coils according to certain embodiments comprise a combination of shape memory polymer and shape memory alloy (e.g., NiTi). In certain such embodiments, an embolic coil comprises a shape memory polymer tube and a shape memory alloy (e.g., NiTi) disposed within the tube. Such embodiments are flexible and allow the size and shape of the shape memory to be further reduced without impacting fatigue properties. In addition, or in other embodiments, shape memory polymers are used with shape memory alloys to create a bi-directional (e.g., capable of expanding and contracting) embolic coil. Bi-directional embolic coils can be created with a wide variety of shape memory material combinations having different characteristics.

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present disclosure. The present disclosure, however, may be practiced without the specific details or with certain alternative equivalent components and methods to those described herein. In other instances, well-known components and methods have not been described in detail so as not to unnecessarily obscure aspects of the present disclosure.

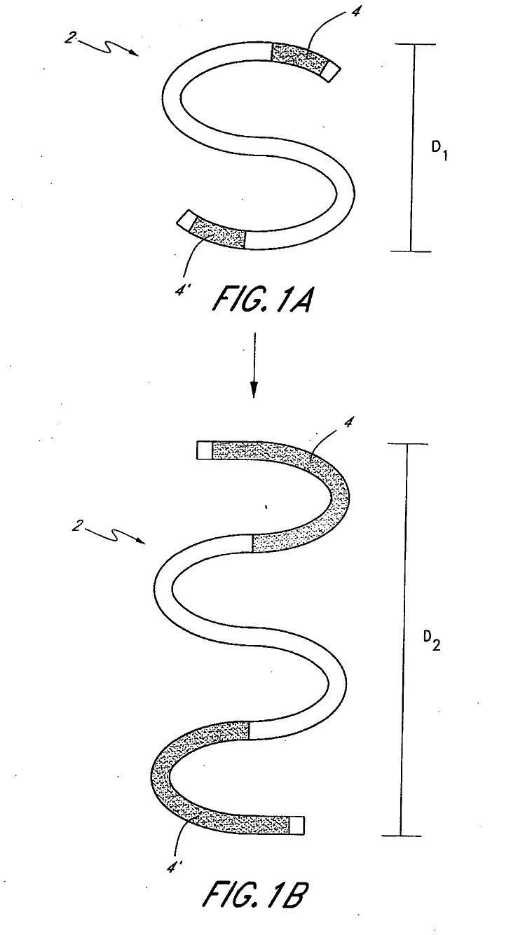

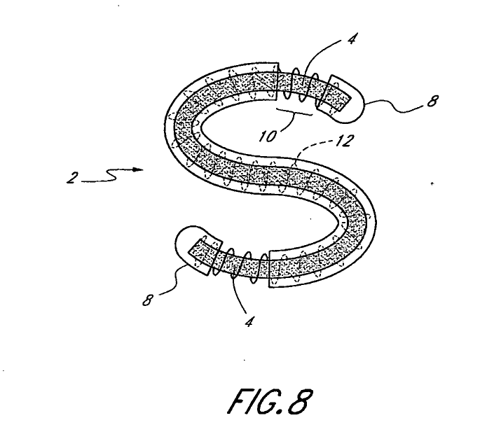

FIG. 1A illustrates a schematic of an adjustable embolic coil 2 according to certain embodiments that can be adjusted after implantation into a patient's body. The embolic coil 2 has a substantially elongate configuration and comprises an elongate member. As used herein, “dimension” is a broad term having its ordinary and customary meaning and includes a measure from a first point to a second point along a line or arc. For example, a dimension may be a circumference, diameter, radius, arc length, width, height, or the like. As another example, a dimension may be a distance between two segments of a coil, an anteroposterior, lateral, rostral-caudal dimension, and the like. The embolic coil is shown in FIG. 1A in a first configuration. While this schematic shows a coil 2 in an “S” configuration, the coil can be any number of different configurations including curvilinear, square, rectangular, triangular, spherical, “figure 8”, a combination of the above, and the like. The coil 2 can be adjustable in one, or multiple dimensions.

In certain embodiments, the nominal length or linear dimension of the embolic coil 2 can be adjusted by 5, 10, 20, 30, 40, 50, 75, 100 percent, or more. However, an artisan will recognize from the disclosure herein that the length or linear dimension of the embolic coil 2 can be adjusted to other sizes depending on the particular application. Indeed, the length or linear dimension of the embolic coil 2 can be configured to fill a sac or lumen with a volume substantially smaller than 1 cc and substantially larger than 20 cc. The initial length of an embolic coil 2 may be 2-50 cm in length, or more, for example, 2 cm, 3 cm, 4 cm, 5 cm, 10 cm, 15 cm, 20 cm, 25 cm, 35 cm, or 50 cm. A coil 2 may have a thickness of about 0.001 to 2 cm, preferably about 0.01 to 0.05 cm, more preferably about 0.02 to 0.04 cm.

The schematic diagram of the embolic coil 2 shown in FIG. 1A depicts an embolic coil with two shape memory members 4, 4′, both in their martensitic state. In certain other embodiments, the coil may include any number of shape memory members 4, 4′, such as one, three, four, five, or more shape memory members. The shape memory member(s) 4, 4′ may be part of, substantially all, or in some embodiments comprise the entire length of the embolic coil. The embolic coil shown in FIG. 1A has an initial linear dimension D1.

In FIG. 1A and certain other embodiments, the embolic coil may comprise a shape memory material that is responsive to changes in temperature and/or exposure to a magnetic field. As discussed above, the shape memory material may include shape memory polymers (e.g., polylactic acid (PLA), polyglycolic acid (PGA)) and/or shape memory alloys (e.g., nickel-titanium) including ferromagnetic shape memory alloys (e.g., Fe—C, Fe—Pd, Fe—Mn—Si, Co—Mn, Fe—Co—Ni—Ti, Ni—Mn—Ga, Ni₂MnGa, Co—Ni—Al). In certain such embodiments, the embolic coil is adjusted in vivo by applying an energy source such as radio frequency energy, X-ray energy, microwave energy, ultrasonic energy such as high intensity focused ultrasound (Hu) energy, light energy, electric field energy, magnetic field energy, combinations of the foregoing, or the like. Preferably, the energy source is applied in a non-invasive manner from outside the body. For example, as discussed above, a magnetic field and/or RF pulses can be applied to the embolic coil within a patient's body with an apparatus external to the patient's body such as is commonly used for magnetic resonance imaging (MRI). However, in other embodiments, the energy source may be applied surgically such as by inserting a catheter into the body and applying the energy through the catheter.

In certain embodiments, the embolic coil comprises a shape memory material that responds to the application of temperature that differs from a nominal ambient temperature, such as the nominal body temperature of 37 degrees Celsius for humans. The embolic coil is configured to respond by starting to contract upon heating the embolic coil above the Aₛ temperature of the shape memory material. In certain such embodiments, the embolic coil may expand or contract by percentage in a range between approximately 5 percent and approximately 50 percent, or more, where the percentage of change is defined as a ratio of the difference between the starting length and finish length divided by the starting length.

The activation temperatures (e.g., temperatures ranging from the As temperature to the A_{f} temperature) at which the embolic coil expands to an elongated linear dimension may be selected and built into the embolic coil such that collateral damage is reduced or eliminated in tissue adjacent the embolic coil during the activation process. Exemplary A_{f} temperatures for the shape memory material of the embolic coil at which substantially maximum expansion occurs are in a range between approximately 38 degrees Celsius and approximately 75 degrees Celsius. In certain embodiments, the A_{f} temperature is in a range between approximately 39 degrees Celsius and approximately 75 degrees Celsius. For some embodiments that include shape memory polymers for the embolic coil, activation temperatures at which the glass transition of the material or substantially maximum contraction occur range between approximately 38 degrees Celsius and approximately 60 degrees Celsius. In other such embodiments, the activation temperature is in a range between approximately 40 degrees Celsius and approximately 59 degrees Celsius.

After implantation of an embolic coil within the sac of an aneurysm, which may be accomplished by any method known in the art, for example, percutaneously via the femoral artery, the embolic coil is preferably activated non-invasively by the application of energy to the patient's body to heat the embolic coil. In certain embodiments, an MRI device is used as discussed above to heat the embolic coil, which then causes the shape memory material of the embolic coil to transform to the austenite phase and remember its contracted configuration. Thus, the length/volume occupied by the embolic coil is increased in vivo without the need for further intervention, such as additional coiling procedures. Standard techniques for focusing the magnetic field from the MRI device onto the embolic coil may be used. For example, a conductive coil can be wrapped around the patient in an area corresponding to the embolic coil. In other embodiments, the shape memory material is activated by exposing it other sources of energy, as discussed above.

FIG. 1B is a schematic illustrating the embolic coil 2 of FIG. 1A with shape memory members 4, 4′ after application of energy to the embolic coil 2. Preferably, the energy is applied noninvasively from a source outside of the patient's body, as described elsewhere in the application. Here, shape memory members 4, 4′ have changed from a martensitic to an austenitic state, allowing the embolic coil 2 to change into a second configuration with a corresponding expanded length D2.

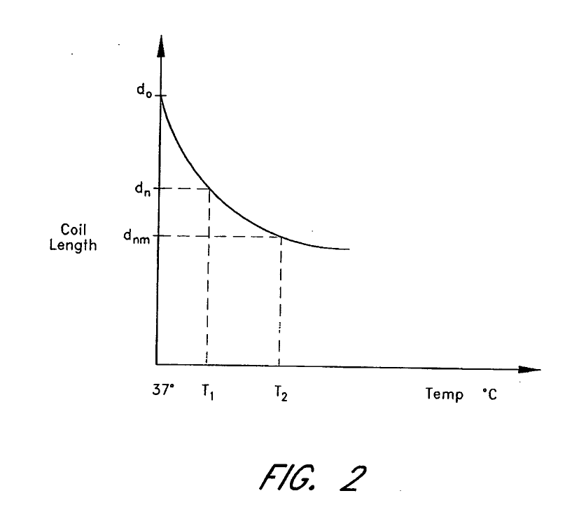

The embolic coil expansion process, either non-invasively or through a catheter, can be carried out all at once or incrementally in small steps at different times in order to achieve the adjustment necessary to produce the desired clinical result. If heating energy is applied such that the temperature of the embolic coil does not reach the A_{f} temperature for substantially maximum transition contraction, partial shape memory transformation and contraction may occur. FIG. 2 graphically illustrates the relationship between the temperature of the embolic coil and the length or linear dimension of the embolic coil according to certain embodiments. At body temperature of approximately 37 degrees Celsius, the length of the embolic coil has a first length d₀. The shape memory material is then increased to a first raised temperature T₁. In response, the length or linear dimension of the embolic coil increases to a second length. The length of the embolic coil can then be increased to a third length dₙₘ by raising the temperature to a second temperature T₂.

As graphically illustrated in FIG. 2, in certain embodiments, the change in length from d₀ to dₙₘ is substantially continuous as the temperature is increased from body temperature to T₂. For example, in certain embodiments a magnetic field of about 2.5 Tesla to about 3.0 Tesla is used to raise the temperature of the embolic coil 2 above the A_{f} temperature to complete the austenite phase and return the embolic coil 2 to the remembered configuration with. However, a lower magnetic field (e.g., 0.5 Tesla) can initially be applied and increased (e.g., in 0.5 Tesla increments) until the desired level of heating and desired contraction of the embolic coil 2 is achieved. In other embodiments, the embolic coil 2 comprises a plurality of shape memory materials with different activation temperatures and the length of embolic coil 2 is increased in steps as the temperature increases.

Whether the shape change is continuous or stepped, the length or linear dimension of the embolic coil 2 can be assessed or monitored during the expansion process to determine the amount of expansion by use of MRI imaging, ultrasound imaging, computed tomography (CT), X-ray or the like. If magnetic energy is being used to activate expansion of the embolic coil 2, for example, MRI imaging techniques can be used that produce a field strength that is lower than that required for activation of the embolic coil 2.

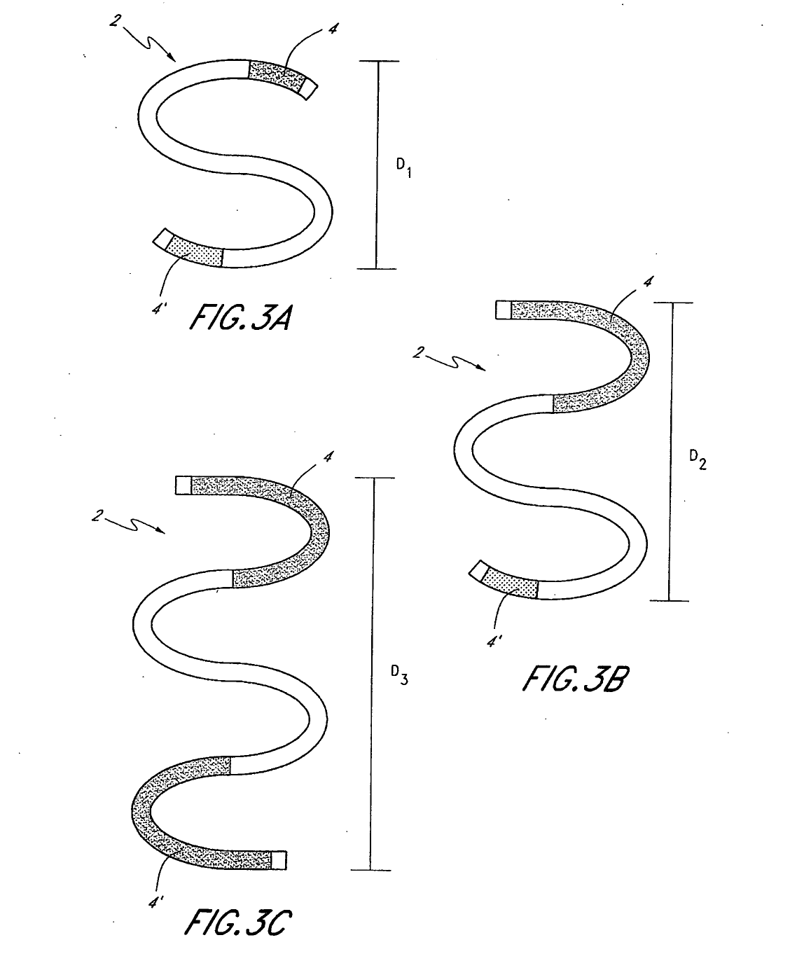

Alternatively, the embolic coil 2 may comprise two or more sections or zones of shape memory material 4, 4′ having different temperature response curves, as in the schematic shown of FIG. 3A. The shape memory response zones may be configured in order to achieve a desired configuration of the embolic coil 2 as a whole when in an expanded state, either fully expanded or partially expanded. For example, the embolic coil 2 may have a first zone or section 4 near one end of the coil 2 and a second zone or section 4′ near the other end of the coil 2. The location of the shape memory zones 4, 4′ within the coil 2 are shown here merely for purposes of illustration and may be located anywhere on the coil 2, such as near or at the midportion of the coil 2. Thus, the first shape memory material zone 4 and the second shape memory zone 4′ can be activated independently such that one transitions to its austenite phase while the other remains in its martensite phase, resulting in expansion of the embolic coil 2 to a second linear dimension D2 and a second configuration, as in FIG. 3B. Activation of both shape memory zones 4 and 4′ results in expansion of the embolic coil 2 to a third linear dimension D3 and a third configuration, as in FIG. 3C. A skilled artisan will appreciate that multiple variations on the number of shape memory zones and coil configurations can be achieved depending on the desired clinical effect. Moreover, the shape memory materials 4, 4′ may also achieve a contracted size and shape after activation, as described elsewhere in the application.

In other embodiments, the shape memory material or materials which are separated into a first temperature response zone 4, and a second temperature response zone 4′. Although the embolic coil 2 is shown with two zones 4, 4′, an artisan will recognize from the disclosure herein that other embodiments may include less or more zones of the same or differing lengths. For example, one embodiment of an embolic coil 2 includes approximately three to approximately eight temperature response zones.

In certain embodiments, the shape memory materials of the various temperature response zones 4, 4′ are selected to have temperature responses and reaction characteristics such that a desired shape and configuration can be achieved in vivo by the application of invasive or non-invasive energy, as discussed above. In addition to general contraction and expansion changes, more subtle changes in shape and configuration for improvement or optimization of aneurysmal filling may be achieved with such embodiments.

According to certain embodiments, the first zone 4 is made from a shape memory material having a first shape memory temperature response. The second zone 4′ is made from a shape memory material having a second shape memory temperature response. In certain embodiments, the two zones 4, 4′ comprise the same shape memory material, such as NiTi alloy or other shape memory material as discussed above, processed to produce the varied temperature response in the respective zones. In other embodiments, the zones may comprise different shape memory materials. Certain embodiments include a combination of shape memory alloys and shape memory polymers in order to achieve the desired results.

According to certain embodiments, FIG. 3C shows the embolic coil 2 after heat activation such that it comprises expanded zones 4, 4′. As schematically shown in FIG. 3C, activation has expanded the zone 4′ so as to increase the axial lengths of the segments of the embolic coil 2 corresponding to those zones. In addition, or in other embodiments, the zones 4, 4′ are configured to contract by a similar percentage instead of expand. In other embodiments, the zones 4, 4′ are configured to each have a different shape memory temperature response such that each segment corresponding to each zone 4, 4′ could be activated sequentially.

FIG. 3C schematically illustrates that the shape memory material zones 4, 4′ have expanded axially (i.e., from their initial configuration as shown by the zones 4, 4′ shown in FIG. 3A). In certain embodiments, a zone 4′ is configured to be thermally activated to remember a shape memory dimension or size upon reaching a temperature in a range between approximately 51 degrees Celsius and approximately 60 degrees Celsius. In certain such embodiments, the zone 4 is configured to respond at temperatures in a range between approximately 41 degrees Celsius and approximately 48 degrees Celsius. Thus, for example, by applying invasive or non-invasive energy, as discussed above, to the embolic coil 2 until the embolic coil 2 reaches a temperature of approximately 41 degrees Celsius to approximately 48 degrees Celsius, the zone 4 will respond by expanding or contracting by virtue of the shape memory mechanism, and the zone 4′ will not.

In certain other embodiments, the zone 4′ is configured to expand or contract by virtue of the shape memory mechanism at a temperature in a range between approximately 50 degrees Celsius and approximately 60 degrees Celsius. In certain such embodiments, the zone 4 is configured to respond at a temperature in a range between approximately 39 degrees Celsius and approximately 45 degrees Celsius.

In certain embodiments, the materials, dimensions and features of the embolic coil 2 and the corresponding zones 4, 4′ have the same or similar features, dimensions or materials as those of the other embolic coil embodiments discussed above. In certain embodiments, the features of the embolic coil 2 are added to the embodiments discussed above.

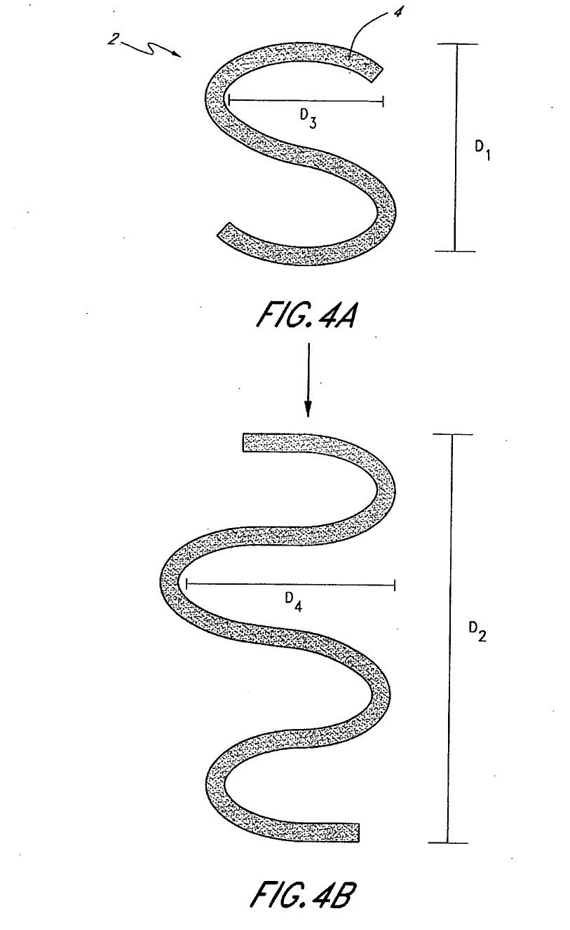

For embodiments of the embolic coil 2 made from a continuous piece of shape memory alloy (e.g., NiTi alloy) or shape memory polymer, such as FIG. 4A, an embolic coil 2 can be activated by the surgical and/or non-invasive application of heating energy by the methods discussed above with regard to other embodiments. For embodiments of the embolic coil 2 made substantially entirely or entirely (as shown in FIG. 4A) from, for example, a continuous piece of ferromagnetic shape memory alloy, the embolic coil 2 can be activated by the non-invasive application of a suitable magnetic field.

The embolic coil 2 has a nominal linear dimension D1 indicated by the schematic FIG. 4A while the coil 2 is in a first configuration. Also, the embolic coil 2 shown has a second nominal linear dimension D3 while the coil 2 is in a first configuration. FIG. 4B is a schematic representation of an embolic coil 2 upon activating the shape memory material 4 of the embolic coil 2 by the application of energy. In certain embodiments as shown, the shape memory material 4 remembers and assumes a second expanded configuration wherein a linear dimension D2 is greater than the nominal linear dimension D1. Moreover, the second linear dimension D4 may be greater than the second nominal linear dimension D3. This may be advantageous, for example, to more optimally occlude an aneurysmal sac by expanding the coil 2 in multiple linear dimensions. In certain other embodiments, the embolic coil 2 is sufficiently malleable when it is implanted into a patient's body that it can be manually adjusted to effectively fill and occlude an aneurysmal sac.

In certain embodiments, upon activating the shape memory material 4 of the embolic coil 2 by the application of energy, the shape memory material 4 remembers and assumes a configuration wherein a linear dimension D2 is less than the nominal linear dimension D1. A contraction in a range between approximately 5-50 percent, or more may be desirable in some embodiments. In certain embodiments, the embolic coil 2 comprises a shape memory NiTi alloy having a linear dimension in a range between approximately 1 cm and approximately 50 cm. In certain such embodiments, the embolic coil 2 can contract or shrink in a range between approximately 5 to 50 percent, or more, where the percentage of contraction is defined as a ratio of the difference between the starting linear dimension and finish linear dimension divided by the starting linear dimension.

As discussed above in relation to FIG. 2, in certain embodiments, a linear dimension, such as D1, of certain embodiments can be altered as a function of the temperature of the embolic coil 2. As also discussed above, in certain such embodiments, the progress of the size change can be measured or monitored in real-time conventional imaging techniques. Energy from conventional imaging devices can also be used to activate the shape memory material and change a linear dimension, such as D1, of the embolic coil 2. In certain embodiments, the features, dimensions and materials of the embolic coil 2 are the same as or similar to the features, dimensions and materials of the embolic coil 2 discussed above. For example, in certain embodiments, the embolic coil 2 comprises a shape memory material 4 that exhibits a two-way shape memory effect when heated and cooled. Thus, the embolic coil 2 in certain such embodiments, can be contracted and expanded.

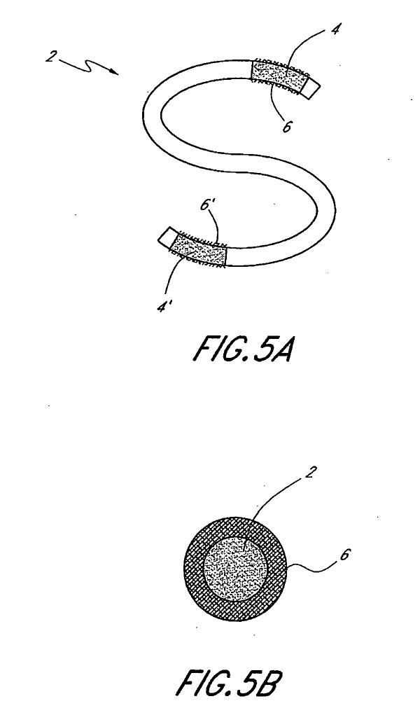

In certain embodiments, the embolic coil 2 comprises an energy absorption enhancement material 6, 6′. As shown in FIG. SA, the energy absorption enhancement material 6, 6′ may cover a portion of the surface of embolic coil 2, multiple discontinuous portions of a coil 2, or the entire coil 2 in other embodiments. As shown in FIG. 5B, the energy absorption enhancement material 6, 6′ may also be coated on the surface of the embolic coil 2 to enhance energy absorption by the embolic coil 2. For embodiments that use energy absorption enhancement material 6, 6′ for enhanced absorption, it may be desirable for the energy absorption enhancement material 6, 6′, a carrier material (not shown) surrounding the energy absorption enhancement material 6, 6′ if there is one, or both to be thermally conductive. Thus, thermal energy from the energy absorption enhancement material 6, 6′ is efficiently transferred to the shape memory material of the embolic coil 2.

As discussed above, the energy absorption enhancement material 6, 6′ may include a material or compound that selectively absorbs a desired heating energy and efficiently converts the non-invasive heating energy to heat which is then transferred by thermal conduction to the embolic coil 2. The energy absorption enhancement material 6, 6′ allows the embolic coil 2 to be actuated and adjusted by the non-invasive application of lower levels of energy and also allows for the use of non-conducting materials, such as shape memory polymers, for the embolic coil 2. For some embodiments, magnetic flux ranging between about 2.5 Tesla and about 3.0 Tesla may be used for activation. By allowing the use of lower energy levels, the energy absorption enhancement material 6, 6′ also reduces thermal damage to nearby tissue. Suitable energy absorption enhancement materials 6, 6′ are discussed above. In some embodiments, an embolic coil 2 may comprise a plurality of different energy enhancement materials, such as one material at 6 and a different material at 6′ that may be especially useful if the coil 2 comprises differing shape memory materials 4, 4′ that change configuration at differing energy exposure levels. In certain other embodiments, an embolic coil 2 may have a first coating 6 and a second coating 6′ each comprise an energy absorption material, such as the energy absorption materials discussed above. In certain such embodiments, the first coating 6 heats when exposed to a first form of energy and the second coating 6′ heats when exposed to a second form of energy. For example, the first coating 6 may heat when exposed to MRI energy and the second coating 6′ may heat when exposed to HIFU energy. As another example, the first coating 6 may heat when exposed to RF energy at a first frequency and the second coating 6′ may heat when exposed to RF energy at a second frequency. Thus, an underlying first shape memory material 4 and a second shape memory material 4′ can be activated independently such that one transitions to its austenite phase while the other remains in its martensite phase, resulting in a change in size or configuration of the embolic coil 2.

In certain embodiments, a linear expansion cycle can be reversed to induce a contraction of the embolic coil 2. Some shape memory alloys, such as NiTi or the like, respond to the application of a temperature below the nominal ambient temperature. After a linear expansion cycle has been performed, the embolic coil 2 is cooled below the Mₛ temperature to start contracting the embolic coil 2. The embolic coil 2 can also be cooled below the M_{f} temperature to finish the transformation to the martensite phase and reverse the linear expansion cycle. As discussed above, certain polymers also exhibit a two-way shape memory effect and can be used to both expand and contract the embolic coil 2 through heating and cooling processes. Cooling can be achieved, for example, by inserting a cool liquid onto or into the embolic coil 2 through a catheter, or by cycling a cool liquid or gas through a catheter placed near the embolic coil 2. Exemplary temperatures for a NiTi embodiment for cooling and reversing a linear expansion cycle range between approximately 20 degrees Celsius and approximately 30 degrees Celsius.

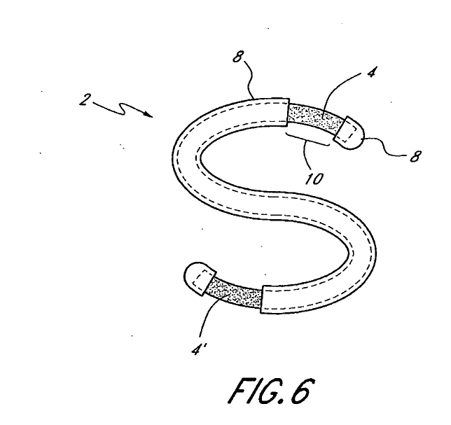

In certain embodiments the embolic coil 2 also comprises a covering 8, shown in the schematic of FIG. 6. The covering 8 may be disposed about the embolic coil 2 to facilitate surgical implantation of the embolic coil 2 in a body structure, such as within an aneurysm. Alternatively, the covering 8 may serve an insulative function in reducing potential thermal, or other damage to neurovascular tissue from energy sources utilized to transform the embolic coil 2 from one configuration to another. In certain embodiments, the covering 8 comprises a suitable biocompatible material such as Dacron®, woven velour, polyurethane, polytetrafluoroethylene (PTFE), heparin-coated fabric, or the like. In other embodiments, the covering 8 comprises a biological material such as bovine or equine pericardium, homograft, autograft, or cell-seeded tissue. The covering 8 may also have a microporous structure to promote, for example, fibrous ingrowth and improved sealing of the aneurysmal cavity. In these or other embodiments, the covering 8 comprises a one or more drugs or other chemicals that may induce coagulation, fibrosis, and the like within the aneurysm. The covering 8 may also comprise an anti-infective agent, such as an antibiotic, that may be useful, for example, for treating a mycotic aneurysm.

The covering 8 may be disposed about the entire length of the embolic coil 2, or selected portions thereof. For example, in certain embodiments, such as shown in FIG. 6, the covering 8 is disposed so as to enclose substantially the entire length except near one or more ends of the embolic coil 2.

In certain embodiments, the embolic coil 2 comprises a rigid material such as stainless steel, titanium, or the like, or a flexible material such as silicon rubber, Dacron®, or the like. In certain such embodiments, after implantation into a patient's body, the length of the embolic coil 2 is adjusted in vivo by inserting a catheter (not shown) into the body and transforming the embolic coil 2 using an energy source attached to the catheter.

In certain embolic coil embodiments, materials used to cover portions of the embolic coil 2 also thermally insulate the shape memory materials so as to increase the time required to activate the shape memory materials through application of thermal energy. Thus, surrounding tissue is exposed to the thermal energy for longer periods of time, which may result in damage to the surrounding tissue. Therefore, in certain embodiments of the invention, thermally conductive materials are configured to penetrate the covering material so as to deliver thermal energy to the shape memory materials such that the time required to activate the shape memory materials is decreased. In other embodiments, an embolic coil 2 may comprise thermal conductive materials without the presence of a covering material.

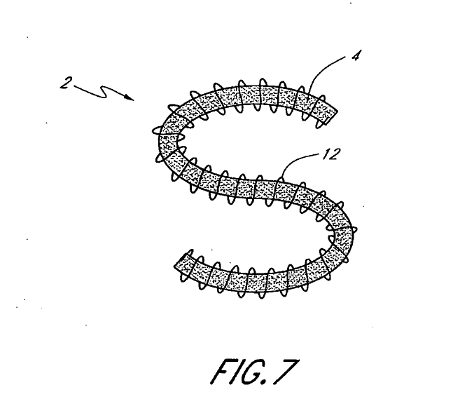

FIG. 7 is a schematic illustrating an embolic coil 2 comprising one or more thermal conductors 12 according to certain embodiments of the invention. In certain embodiments, the thermal conductors 12 comprise a thin (e.g., having a thickness in a range between approximately 0.002 inches and approximately 0.015 inches) wire wrapped around the outside of the embolic coil 2. In embodiments where an insulative covering 8 is present along with the one or more thermal conductors 12, the thermal conductors 12 may penetrating the covering 8 at one or more locations so as to transfer externally applied heat energy to the shape memory material portions 4, 4′ of the embolic coil 2. In certain embodiments, the thermal conductor 12 wraps around the embolic coil 2 one or more times. In other embodiments with an insulative covering 8, such as the schematic diagram of FIG. 8, the thermal conductor 12 penetrates the insulative covering 8, passes around the shape memory material 4, and exits the insulative covering 8. In certain embodiments, the thermal conductor 12 physically contacts the shape memory material 4. However, in other embodiments, the thermal conductor 12 does not physically contact the shape memory material portion 4 but passes sufficiently close to the shape memory material 4 so as to decrease the time required to activate the shape memory material 4. Thus, the potential for thermal damage to surrounding tissue is reduced.

In alternative embodiments, the thermal conductor 12 wraps around the insulative covering 8 one or more times, penetrates the insulative covering 8, passes around the shape memory material 4 two or more times, and exits the insulative covering 8. By passing around the shape memory material 4 two or more times, the thermal conductor 12 concentrates more energy in the area of the shape memory material 4 as described above. Again, the thermal conductor 12 may or may not physically contact the shape memory material 4.

In yet other embodiments, the thermal conductor 12 wraps around the insulative covering one or more times and passes through the insulative covering 8 two or more times. Thus, portions of the thermal conductor 12 are disposed proximate the shape memory material 4 so as to transfer heat energy thereto. Again, the thermal conductor 12 may or may not physically contact the shape memory material 4. An artisan will recognize from the disclosure herein that one or more of the embodiments described above can be combined and that the thermal conductor 12 can be configured to penetrate the insulative covering 8 in other ways in accordance with the invention so as to transfer heat to the shape memory material 4.

Thus, thermal energy can be quickly transferred to the embolic coil 2 to reduce the amount of energy required to activate the shape memory material 4 and to reduce thermal damage to the patient's surrounding tissue.

In yet another embodiment, after the coil is packed within an aneurysm in a first procedure, the aneurysm may be adjusted at a later time by delivering a catheter to the coil 2 site during a second procedure, preferably percutaneously, and delivering energy from a catheter configured to deliver such energy within the body to cause the coil 2 to change configuration, using any of the types of energy described above, such as RF energy, acoustic energy, and the like.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A method for treating an aneurysm within a patient, said method comprising:
providing an embolic coil, comprising a shape memory material and having a first size of a dimension of said coil in a first configuration and a second size of said dimension of said coil in a second configuration;
packing said embolic coil, while said coil is in said first configuration, within an aneurysm; and
applying energy from outside the patient's body to said shape memory material of said embolic coil located inside said patient's body, thereby changing said embolic coil from said first configuration to said second configuration.

2. The method of claim 1, wherein applying said energy to said embolic coil comprises heating said shape memory material of said embolic coil to a predetermined temperature, wherein said shape memory material changes shape in response to being heated to said predetermined temperature.

3. The method of claim 2, wherein said heating of said shape memory material comprises applying said energy to an energy absorption material in thermal communication with said shape memory material.

4. The method of claim 2, wherein said heating of said shape memory material comprises applying said energy to an electrically conductive material in thermal communication with said shape memory material, wherein said energy produces a current in said electrically conductive material.

5. The method of claim 1, wherein applying said energy to said embolic coil comprises generating a magnetic field outside said patient's body, wherein said magnetic field is configured to change the shape of a shape memory material of said embolic coil.

6. The method of claim 5, wherein said shape memory material comprises a ferromagnetic material.

7. The method of claim 1, wherein applying said energy comprises generating magnetic field energy,

8. The method of claim 1, wherein applying said energy comprises generating electromagnetic energy.

9. The method of claim 1, wherein applying said energy comprises generating mechanical energy.

10. The method of claim 1, wherein applying said energy comprises generating acoustic energy.

11. The method of claim 10, wherein said acoustic energy is focused ultrasound energy.

12. The method of claim 10, wherein said acoustic energy comprises high-intensity focused ultrasound energy.

13. The method of claim 12, further comprising generating said high-intensity focused ultrasound energy with a handheld device.

14. The method of claim 8, further comprising generating said electromagnetic energy using a handheld device.

15. The method of claim 7, further comprising generating said magnetic field using a magnetic resonance device.

16. The method of claim 15, further comprising imaging said embolic coil with said magnetic resonance device.

17. The method of claim 1, further comprising non-invasively monitoring the sizes of said embolic coil before and after said embolic coil changes from said first configuration to said second configuration.

18. The method of claim 17, wherein non-invasively monitoring the sizes of said embolic coil comprises operating a monitoring device comprising at least one of a magnetic resonance imaging device, an ultrasound imaging device, a computed tomography device, and an X-ray device.

19. The method of claim 1, wherein said second size is larger than said first size.

20. The method of claim 1, wherein said second size is smaller than said first size.

21. The method of claim 1, further comprising changing said embolic coil from said second size to a third size of said dimension in a third configuration.

22. The method of claim 21, wherein said third size is less than said second size.

23. The method of claim 22, wherein said third size is larger than said second size.

24. The method of claim 1, wherein the dimension is a linear dimension.

25. The method of claim 1, wherein the embolic coil further comprises an energy-absorbing material over at least a portion of said coil.

26. The method of claim 1, wherein said coil further comprises a covering extending over at least a portion of said coil.

27. An adjustable embolic coil, for treating an aneurysm of a patient, comprising:
a shape memory material;
a first size of a dimension of said coil when said coil is in a first configuration;
a second size of said dimension of said coil when said coil is in a second configuration;
said coil being changeable from the first configuration to the second configuration in response to an application of energy from outside the patient's body to said shape memory material of said embolic coil, when said coil is located inside said patient's body.

28. The embolic coil of claim 27, said coil further comprising a first energy-absorbing material extending over at least a portion of said coil.

29. The embolic coil of claim 27, said embolic coil further having a third size of a dimension of said coil in a third configuration, said coil being changeable from the second configuration to a third configuration by applying energy to a shape memory material of said embolic coil.

30. The embolic coil of claim 28, wherein the first energy-absorbing material absorbs electromagnetic energy.

31. The embolic coil of claim 30, wherein the coil further comprises a second energy-absorbing material.

32. The embolic coil of claim 27, further comprising a covering that at least partially surrounds the shape memory material.

33. The embolic coil of claim 32; wherein the covering is discontinuous along said embolic coil.

34. The embolic coil of claim 32, where the covering has insulative properties.

35. The embolic coil of claim 32, where the covering further comprises a therapeutic agent.

36. The embolic coil of claim 32, further comprising a thermal conductor coupled the coil.
